(19) United States
(12) Patent Application Publication
(10) Pub. No.: US 2023/0128104 A1
(43) Pub. Date: Apr. 27, 2023

(54) ACCESSIBILITY SYSTEM FOR ASSISTING A USER IN INTERACTING WITH A VEHICLE

(71) Applicant: Motional AD LLC, Boston, MA (US)

(72) Inventor: Eryk Brian Nice, Medford, MA (US)

(21) Appl. No.: 17/509,333

(22) Filed: Oct. 25, 2021

(51) Int. Cl. B60Q 9/00; G06T 7/70; G06T 7/50; G08B 3/10; G06F 3/16

(52) U.S. Cl. CPC B60Q 9/008; G06T 7/70; G06T 7/50; G08B 3/10; G06F 3/16; G06T 2207/30196; G06T 2207/30248; G06T 2207/10016; G01S 13/931

(57) ABSTRACT

Provided are methods for assisting a user in interaction with a vehicle. The methods can include obtaining sensor data representing a user; determining at least one of: (i) a distance between a body part of the user and an object associated with the vehicle, or (ii) a direction from the body part of the user to the object; and causing, by the accessibility system, at least one notification to be presented to the user, where the least one notification indicates at least one of: (i) the distance between the body part of the user and the object, or (ii) the direction from the body part of the user to the object. Systems and computer program products are also provided.

### BACKGROUND

Vehicles can be used to transport people from one location to another. For example, a person can enter the passenger compartment of a vehicle, and use the vehicle to travel to a destination (e.g., by manually driving the vehicle and/or instructing an autonomous system of the vehicle to navigate the vehicle to the destination).

In some implementations, a person may physically interact with a vehicle. For example, a person can manipulate a door handle to open a door of the vehicle. As another example, a person can manipulate a control mechanism (e.g., a button, a knob, wheel, a lever, etc.) to input commands to the vehicle.

### BRIEF DESCRIPTION OF THE FIGURES

FIG. 1 is an example environment in which a vehicle including one or more components of an autonomous system can be implemented;

FIG. 2 is a diagram of one or more systems of a vehicle including an autonomous system;

FIG. 3 is a diagram of components of one or more devices and/or one or more systems of FIGS. 1 and 2;

FIG. 4 is a diagram of an example accessibility system.

FIGS. 5A and 5B are diagrams showing an example operation of an accessibility system.

FIG. 6 is a diagram showing another example operation of an accessibility system.

FIG. 7A is a diagram of an implementation of a neural network;

FIGS. 7B and 7C are diagram illustrating example operation of a neural network;

FIG. 8 is a flowchart of a process for detecting objects within a vehicle.

### DETAILED DESCRIPTION

In the following description numerous specific details are set forth in order to provide a thorough understanding of the present disclosure for the purposes of explanation. It will be apparent, however, that the embodiments described by the present disclosure can be practiced without these specific details. In some instances, well-known structures and devices are illustrated in block diagram form in order to avoid unnecessarily obscuring aspects of the present disclosure.

Specific arrangements or orderings of schematic elements, such as those representing systems, devices, modules, instruction blocks, data elements, and/or the like are illustrated in the drawings for ease of description. However, it will be understood by those skilled in the art that the specific ordering or arrangement of the schematic elements in the drawings is not meant to imply that a particular order or sequence of processing, or separation of processes, is required unless explicitly described as such. Further, the inclusion of a schematic element in a drawing is not meant to imply that such element is required in all embodiments or that the features represented by such element may not be included in or combined with other elements in some embodiments unless explicitly described as such.

Further, where connecting elements such as solid or dashed lines or arrows are used in the drawings to illustrate a connection, relationship, or association between or among two or more other schematic elements, the absence of any such connecting elements is not meant to imply that no connection, relationship, or association can exist. In other words, some connections, relationships, or associations between elements are not illustrated in the drawings so as not to obscure the disclosure. In addition, for ease of illustration, a single connecting element can be used to represent multiple connections, relationships or associations between elements. For example, where a connecting element represents communication of signals, data, or instructions (e.g., “software instructions”), it should be understood by those skilled in the art that such element can represent one or multiple signal paths (e.g., a bus), as may be needed, to affect the communication.

Although the terms first, second, third, and/or the like are used to describe various elements, these elements should not be limited by these terms. The terms first, second, third, and/or the like are used only to distinguish one element from another. For example, a first contact could be termed a second contact and, similarly, a second contact could be termed a first contact without departing from the scope of the described embodiments. The first contact and the second contact are both contacts, but they are not the same contact.

The terminology used in the description of the various described embodiments herein is included for the purpose of describing particular embodiments only and is not intended to be limiting. As used in the description of the various described embodiments and the appended claims, the singular forms “a,” “an” and “the” are intended to include the plural forms as well and can be used interchangeably with “one or more” or “at least one,” unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this description specify the presence of stated features, integers, steps, operations, elements, and/or components, but do not preclude the presence or addition of one or more other features, integers, steps, operations, elements, components, and/or groups thereof.

As used herein, the terms “communication” and “communicate” refer to at least one of the reception, receipt, transmission, transfer, provision, and/or the like of information (or information represented by, for example, data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some embodiments, a message may refer to a network packet (e.g., a data packet and/or the like) that includes data.

As used herein, the term “if” is, optionally, construed to mean “when”, “upon”, “in response to determining,” “in response to detecting,” and/or the like, depending on the context. Similarly, the phrase “if it is determined” or “if [a stated condition or event] is detected” is, optionally, construed to mean “upon determining,” “in response to determining,” “upon detecting [the stated condition or event],” “in response to detecting [the stated condition or event],” and/or the like, depending on the context. Also, as used herein, the terms “has”, “have”, “having”, or the like are intended to be open-ended terms. Further, the phrase “based on” is intended to mean “based at least partially on” unless explicitly stated otherwise.

Reference will now be made in detail to embodiments, examples of which are illustrated in the accompanying drawings. In the following detailed description, numerous specific details are set forth in order to provide a thorough understanding of the various described embodiments. However, it will be apparent to one of ordinary skill in the art that the various described embodiments can be practiced without these specific details. In other instances, well-known methods, procedures, components, circuits, and networks have not been described in detail so as not to unnecessarily obscure aspects of the embodiments.

General Overview

In some aspects and/or embodiments, the systems, methods, and computer program products described herein include and/or implement techniques for assisting a user in interacting with a vehicle. In an example implementation, a vehicle includes an accessibility system that detects the location of a user's hand, and provides feedback to the user regarding the distance between the user's hand and an object of interest and/or the location of the user's hand relative to the location of the object. This enables the user to move his hand towards the object, such that she can physically interact with the object.

As an illustrative example, an accessibility system can determine that a user is attempting to enter a vehicle, and in response, generate notifications that assist the user in locating and grasping a door handle of that vehicle. For instance, the accessibility system can receive sensor data (e.g., images, videos, radar data, LiDAR data, ultrasonic sensor data, etc.), and based on the sensor data, determine the location of the user's hand relative to the door handle. Upon determining that the user is moving his hand towards the door handle, the accessibility system can generate an auditory signal indicating that the user is moving his hand in the correct direction (e.g., getting “warmer”). Further, upon determining that the user is moving his hand away the door handle, the accessibility system can generate an auditory signal indicating that the user is moving his hand in the wrong direction (e.g., getting “colder”).

In general, the accessibility system can assist the user in interacting with any component or control mechanism of a vehicle, such as a door handle, a button, a switch, a latch, a knob, a seat belt, etc. Further, the accessibility system can guide a user towards an object located in the vehicle, such as a personal item left within the passenger compartment of the vehicle.

Some of the advantages of these techniques include enabling users to interact with a vehicle more easily (e.g., compared to interacting with a vehicle that does not include an accessibility system described herein). As an example, an accessibility system can enable visually impaired users to enter and exit a vehicle more easily, despite the user having difficulty visually ascertaining the location of door handle or other mechanisms of the vehicle. As another example, an accessibility system can guide users to the location of certain controls of a vehicle, despite the user being unfamiliar with the physical configuration of the vehicle. As another example, an accessibility system can guide users to the location of misplaced objects within the vehicle.

These techniques may be particularly advantageous in vehicles that are shared among several different users (e.g., an autonomous vehicle used in a ride-sharing service). For example, the techniques described herein can reduce delays associated with users entering, operating, and/or exiting vehicles. Accordingly, the vehicles can be operated in a more efficient manner (e.g., compared to vehicles that do not include an accessibility system described herein).

In some embodiments, the techniques described herein can be implemented within vehicles, such as vehicles having autonomous systems (e.g., autonomous vehicles) and/or vehicles that do not have autonomous systems.

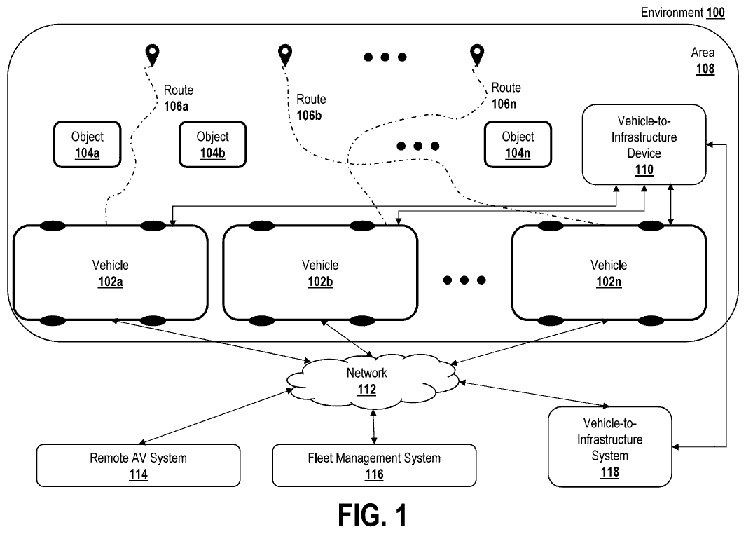

Referring now to FIG. 1, illustrated is example environment 100 in which vehicles that include autonomous systems, as well as vehicles that do not, are operated. As illustrated, environment 100 includes vehicles 102a-102n, objects 104a-104n, routes 106a-106n, area 108, vehicle-to-infrastructure (V2I) device 110, network 112, remote autonomous vehicle (AV) system 114, fleet management system 116, and V2I system 118. Vehicles 102a-102n, vehicle-to-infrastructure (V2I) device 110, network 112, autonomous vehicle (AV) system 114, fleet management system 116, and V2I system 118 interconnect (e.g., establish a connection to communicate and/or the like) via wired connections, wireless connections, or a combination of wired or wireless connections. In some embodiments, objects 104a-104n interconnect with at least one of vehicles 102a-102n, vehicle-to-infrastructure (V2I) device 110, network 112, autonomous vehicle (AV) system 114, fleet management system 116, and V2I system 118 via wired connections, wireless connections, or a combination of wired or wireless connections.

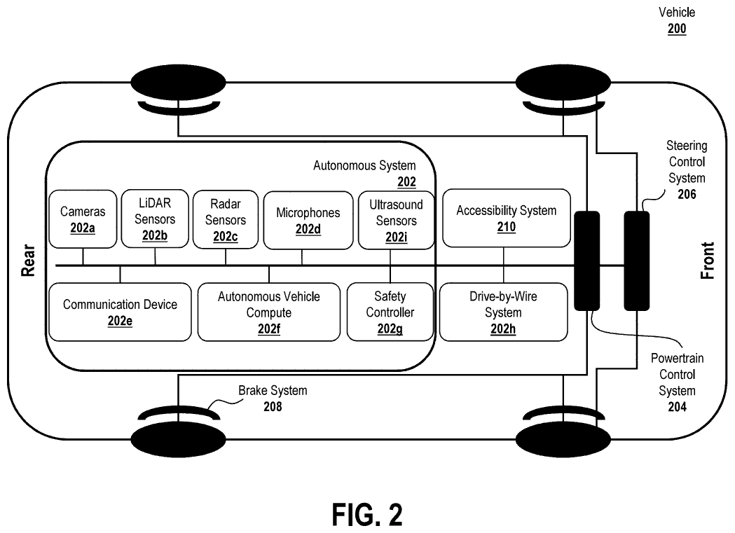

Vehicles 102a-102n (referred to individually as vehicle 102 and collectively as vehicles 102) include at least one device configured to transport goods and/or people. In some embodiments, vehicles 102 are configured to be in communication with V2I device 110, remote AV system 114, fleet management system 116, and/or V2I system 118 via network 112. In some embodiments, vehicles 102 include cars, buses, trucks, trains, and/or the like. In some embodiments, vehicles 102 are the same as, or similar to, vehicles 200, described herein (see FIG. 2). In some embodiments, a vehicle 200 of a set of vehicles 200 is associated with an autonomous fleet manager. In some embodiments, vehicles 102 travel along respective routes 106a-106n (referred to individually as route 106 and collectively as routes 106), as described herein. In some embodiments, one or more vehicles 102 include an autonomous system (e.g., an autonomous system that is the same as or similar to autonomous system 202).

Objects 104a-104n (referred to individually as object 104 and collectively as objects 104) include, for example, at least one vehicle, at least one pedestrian, at least one cyclist, at least one structure (e.g., a building, a sign, a fire hydrant, etc.), and/or the like. Each object 104 is stationary (e.g., located at a fixed location for a period of time) or mobile (e.g., having a velocity and associated with at least one trajectory). In some embodiments, objects 104 are associated with corresponding locations in area 108.

Routes 106a-106n (referred to individually as route 106 and collectively as routes 106) are each associated with (e.g., prescribe) a sequence of actions (also known as a trajectory) connecting states along which an AV can navigate. Each route 106 starts at an initial state (e.g., a state that corresponds to a first spatiotemporal location, velocity, and/or the like) and a final goal state (e.g., a state that corresponds to a second spatiotemporal location that is different from the first spatiotemporal location) or goal region (e.g., a subspace of acceptable states (e.g., terminal states)). In some embodiments, the first state includes a location at which an individual or individuals are to be picked-up by the AV and the second state or region includes a location or locations at which the individual or individuals picked-up by the AV are to be dropped-off. In some embodiments, routes 106 include a plurality of acceptable state sequences (e.g., a plurality of spatiotemporal location sequences), the plurality of state sequences associated with (e.g., defining) a plurality of trajectories. In an example, routes 106 include only high level actions or imprecise state locations, such as a series of connected roads dictating turning directions at roadway intersections. Additionally, or alternatively, routes 106 may include more precise actions or states such as, for example, specific target lanes or precise locations within the lane areas and targeted speed at those positions. In an example, routes 106 include a plurality of precise state sequences along the at least one high level action sequence with a limited lookahead horizon to reach intermediate goals, where the combination of successive iterations of limited horizon state sequences cumulatively correspond to a plurality of trajectories that collectively form the high level route to terminate at the final goal state or region.

Area 108 includes a physical area (e.g., a geographic region) within which vehicles 102 can navigate. In an example, area 108 includes at least one state (e.g., a country, a province, an individual state of a plurality of states included in a country, etc.), at least one portion of a state, at least one city, at least one portion of a city, etc. In some embodiments, area 108 includes at least one named thoroughfare (referred to herein as a “road”) such as a highway, an interstate highway, a parkway, a city street, etc. Additionally, or alternatively, in some examples area 108 includes at least one unnamed road such as a driveway, a section of a parking lot, a section of a vacant and/or undeveloped lot, a dirt path, etc. In some embodiments, a road includes at least one lane (e.g., a portion of the road that can be traversed by vehicles 102). In an example, a road includes at least one lane associated with (e.g., identified based on) at least one lane marking.

Vehicle-to-Infrastructure (V2I) device 110 (sometimes referred to as a Vehicle-to-Infrastructure (V2X) device) includes at least one device configured to be in communication with vehicles 102 and/or V2I infrastructure system 118. In some embodiments, V2I device 110 is configured to be in communication with vehicles 102, remote AV system 114, fleet management system 116, and/or V2I system 118 via network 112. In some embodiments, V2I device 110 includes a radio frequency identification (RFID) device, signage, cameras (e.g., two-dimensional (2D) and/or three-dimensional (3D) cameras), lane markers, streetlights, parking meters, etc. In some embodiments, V2I device 110 is configured to communicate directly with vehicles 102. Additionally, or alternatively, in some embodiments V2I device 110 is configured to communicate with vehicles 102, remote AV system 114, and/or fleet management system 116 via V2I system 118. In some embodiments, V2I device 110 is configured to communicate with V2I system 118 via network 112.

Network 112 includes one or more wired and/or wireless networks. In an example, network 112 includes a cellular network (e.g., a long term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN), a private network, an ad hoc network, an intranet, the Internet, a fiber optic-based network, a cloud computing network, etc., a combination of some or all of these networks, and/or the like.

Remote AV system 114 includes at least one device configured to be in communication with vehicles 102, V2I device 110, network 112, remote AV system 114, fleet management system 116, and/or V2I system 118 via network 112. In an example, remote AV system 114 includes a server, a group of servers, and/or other like devices. In some embodiments, remote AV system 114 is co-located with the fleet management system 116. In some embodiments, remote AV system 114 is involved in the installation of some or all of the components of a vehicle, including an autonomous system, an autonomous vehicle compute, software implemented by an autonomous vehicle compute, and/or the like. In some embodiments, remote AV system 114 maintains (e.g., updates and/or replaces) such components and/or software during the lifetime of the vehicle.

Fleet management system 116 includes at least one device configured to be in communication with vehicles 102, V2I device 110, remote AV system 114, and/or V2I infrastructure system 118. In an example, fleet management system 116 includes a server, a group of servers, and/or other like devices. In some embodiments, fleet management system 116 is associated with a ridesharing company (e.g., an organization that controls operation of multiple vehicles (e.g., vehicles that include autonomous systems and/or vehicles that do not include autonomous systems) and/or the like).

In some embodiments, V2I system 118 includes at least one device configured to be in communication with vehicles 102, V2I device 110, remote AV system 114, and/or fleet management system 116 via network 112. In some examples, V2I system 118 is configured to be in communication with V2I device 110 via a connection different from network 112. In some embodiments, V2I system 118 includes a server, a group of servers, and/or other like devices. In some embodiments, V2I system 118 is associated with a municipality or a private institution (e.g., a private institution that maintains V2I device 110 and/or the like).

The number and arrangement of elements illustrated in FIG. 1 are provided as an example. There can be additional elements, fewer elements, different elements, and/or differently arranged elements, than those illustrated in FIG. 1. Additionally, or alternatively, at least one element of environment 100 can perform one or more functions described as being performed by at least one different element of FIG. 1. Additionally, or alternatively, at least one set of elements of environment 100 can perform one or more functions described as being performed by at least one different set of elements of environment 100.

Referring now to FIG. 2, vehicle 200 includes autonomous system 202, powertrain control system 204, steering control system 206, brake system 208, and an accessibility system 210. In some embodiments, vehicle 200 is the same as or similar to vehicle 102 (see FIG. 1). In some embodiments, vehicle 102 have autonomous capability (e.g., implement at least one function, feature, device, and/or the like that enable vehicle 200 to be partially or fully operated without human intervention including, without limitation, fully autonomous vehicles (e.g., vehicles that forego reliance on human intervention), highly autonomous vehicles (e.g., vehicles that forego reliance on human intervention in certain situations), and/or the like). For a detailed description of fully autonomous vehicles and highly autonomous vehicles, reference may be made to SAE International's standard J3016: Taxonomy and Definitions for Terms Related to On-Road Motor Vehicle Automated Driving Systems, which is incorporated by reference in its entirety. In some embodiments, vehicle 200 is associated with an autonomous fleet manager and/or a ridesharing company.

Autonomous system 202 includes a sensor suite that includes one or more devices such as cameras 202a, LiDAR sensors 202b, radar sensors 202c, microphones 202d, and ultrasound sensors 202i. In some embodiments, autonomous system 202 can include more or fewer devices and/or different devices (e.g., ultrasonic sensors, inertial sensors, GPS receivers (discussed below), odometry sensors that generate data associated with an indication of a distance that vehicle 200 has traveled, and/or the like). In some embodiments, autonomous system 202 uses the one or more devices included in autonomous system 202 to generate data associated with environment 100, described herein. The data generated by the one or more devices of autonomous system 202 can be used by one or more systems described herein to observe the environment (e.g., environment 100) in which vehicle 200 is located. In some embodiments, autonomous system 202 includes communication device 202e, autonomous vehicle compute 202f, and drive-by-wire (DBW) system 202h.

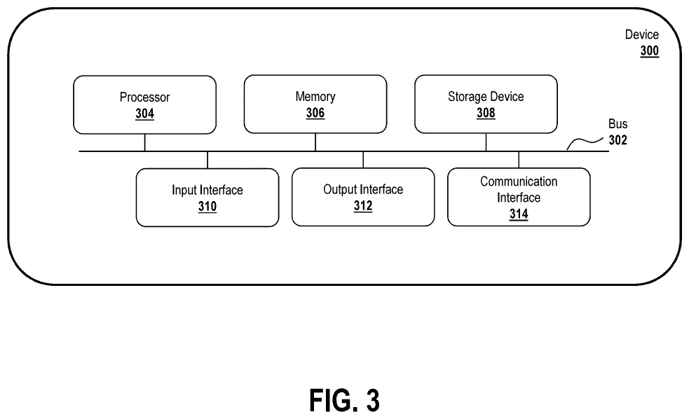

Cameras 202a include at least one device configured to be in communication with communication device 202e, autonomous vehicle compute 202f, and/or safety controller 202g via a bus (e.g., a bus that is the same as or similar to bus 302 of FIG. 3). Cameras 202a include at least one camera (e.g., a digital camera using a light sensor such as a charge-coupled device (CCD), a thermal camera, an infrared (IR) camera, an event camera, and/or the like) to capture images including physical objects (e.g., cars, buses, curbs, people, and/or the like). In some embodiments, camera 202a generates camera data as output. In some examples, camera 202a generates camera data that includes image data associated with an image. In this example, the image data may specify at least one parameter (e.g., image characteristics such as exposure, brightness, etc., an image timestamp, and/or the like) corresponding to the image. In such an example, the image may be in a format (e.g., RAW, JPEG, PNG, and/or the like). In some embodiments, camera 202a includes a plurality of independent cameras configured on (e.g., positioned on) a vehicle to capture images for the purpose of stereopsis (stereo vision). In some examples, camera 202a includes a plurality of cameras that generate image data and transmit the image data to autonomous vehicle compute 202f and/or a fleet management system (e.g., a fleet management system that is the same as or similar to fleet management system 116 of FIG. 1). In such an example, autonomous vehicle compute 202f determines depth to one or more objects in a field of view of at least two cameras of the plurality of cameras based on the image data from the at least two cameras. In some embodiments, cameras 202a is configured to capture images of objects within a distance from cameras 202a (e.g., up to 100 meters, up to a kilometer, and/or the like). Accordingly, cameras 202a include features such as sensors and lenses that are optimized for perceiving objects that are at one or more distances from cameras 202a.

In an embodiment, camera 202a includes at least one camera configured to capture one or more images associated with one or more traffic lights, street signs and/or other physical objects that provide visual navigation information. In some embodiments, camera 202a generates traffic light data associated with one or more images. In some examples, camera 202a generates TLD data associated with one or more images that include a format (e.g., RAW, JPEG, PNG, and/or the like). In some embodiments, camera 202a that generates TLD data differs from other systems described herein incorporating cameras in that camera 202a can include one or more cameras with a wide field of view (e.g., a wide-angle lens, a fish-eye lens, a lens having a viewing angle of approximately 120 degrees or more, and/or the like) to generate images about as many physical objects as possible.

Laser Detection and Ranging (LiDAR) sensors 202b include at least one device configured to be in communication with communication device 202e, autonomous vehicle compute 202f, and/or safety controller 202g via a bus (e.g., a bus that is the same as or similar to bus 302 of FIG. 3). LiDAR sensors 202b include a system configured to transmit light from a light emitter (e.g., a laser transmitter). Light emitted by LiDAR sensors 202b include light (e.g., infrared light and/or the like) that is outside of the visible spectrum. In some embodiments, during operation, light emitted by LiDAR sensors 202b encounters a physical object (e.g., a vehicle) and is reflected back to LiDAR sensors 202b. In some embodiments, the light emitted by LiDAR sensors 202b does not penetrate the physical objects that the light encounters. LiDAR sensors 202b also include at least one light detector which detects the light that was emitted from the light emitter after the light encounters a physical object. In some embodiments, at least one data processing system associated with LiDAR sensors 202b generates an image (e.g., a point cloud, a combined point cloud, and/or the like) representing the objects included in a field of view of LiDAR sensors 202b. In some examples, the at least one data processing system associated with LiDAR sensor 202b generates an image that represents the boundaries of a physical object, the surfaces (e.g., the topology of the surfaces) of the physical object, and/or the like. In such an example, the image is used to determine the boundaries of physical objects in the field of view of LiDAR sensors 202b.

Radio Detection and Ranging (radar) sensors 202c include at least one device configured to be in communication with communication device 202e, autonomous vehicle compute 202f, and/or safety controller 202g via a bus (e.g., a bus that is the same as or similar to bus 302 of FIG. 3). Radar sensors 202c include a system configured to transmit radio waves (either pulsed or continuously). The radio waves transmitted by radar sensors 202c include radio waves that are within a predetermined spectrum. In some embodiments, during operation, radio waves transmitted by radar sensors 202c encounter a physical object and are reflected back to radar sensors 202c. In some embodiments, the radio waves transmitted by radar sensors 202c are not reflected by some objects. In some embodiments, at least one data processing system associated with radar sensors 202c generates signals representing the objects included in a field of view of radar sensors 202c. For example, the at least one data processing system associated with radar sensor 202c generates an image that represents the boundaries of a physical object, the surfaces (e.g., the topology of the surfaces) of the physical object, and/or the like. In some examples, the image is used to determine the boundaries of physical objects in the field of view of radar sensors 202c.

Microphones 202d includes at least one device configured to be in communication with communication device 202e, autonomous vehicle compute 202f, and/or safety controller 202g via a bus (e.g., a bus that is the same as or similar to bus 302 of FIG. 3). Microphones 202d include one or more microphones (e.g., array microphones, external microphones, and/or the like) that capture audio signals and generate data associated with (e.g., representing) the audio signals. In some examples, microphones 202d include transducer devices and/or like devices. In some embodiments, one or more systems described herein can receive the data generated by microphones 202d and determine a position of an object relative to vehicle 200 (e.g., a distance and/or the like) based on the audio signals associated with the data.

Ultrasound sensors 202i include at least one device configured to be in communication with communication device 202e, autonomous vehicle compute 202f, and/or safety controller 202g via a bus (e.g., a bus that is the same as or similar to bus 302 of FIG. 3). Ultrasound sensors 202i include a system configured to transmit ultrasonic sound pulses (either pulsed or continuously), such as a ultrasonic transducer system. The ultrasonic sound pulses transmitted by ultrasound sensors 202i include sound pulses waves that are within a predetermined frequency range. In some embodiments, during operation, sound pulses transmitted by ultrasound sensors 202i encounter a physical object and are reflected back to ultrasound sensors 202i (e.g., in the form of sound echoes). In some embodiments, the sound pulses transmitted by ultrasound sensors 202i are not reflected by some objects. In some embodiments, at least one data processing system associated with ultrasound sensors 202i generates signals representing the objects included in a field of view of ultrasound sensors 202i. For example, the at least one data processing system associated with ultrasound sensors 202i generates range information representing the distance between the ultrasound sensors 202i and a physical object. As another example, the at least one data processing system associated with ultrasound sensors 202i generates an image that represents the boundaries of a physical object, the surfaces (e.g., the topology of the surfaces) of the physical object, and/or the like. In some examples, the image is used to determine the boundaries of physical objects in the field of view of radar ultrasound sensors 202i.

Communication device 202e include at least one device configured to be in communication with cameras 202a, LiDAR sensors 202b, radar sensors 202c, microphones 202d, autonomous vehicle compute 202f, safety controller 202g, DBW system 202h, ultrasound sensors 202i, and/or the accessibility system 210. For example, communication device 202e may include a device that is the same as or similar to communication interface 314 of FIG. 3. In some embodiments, communication device 202e includes a vehicle-to-vehicle (V2V) communication device (e.g., a device that enables wireless communication of data between vehicles).

Autonomous vehicle compute 202f include at least one device configured to be in communication with cameras 202a, LiDAR sensors 202b, radar sensors 202c, microphones 202d, communication device 202e, safety controller 202g, DBW system 202h, and/or ultrasound sensors 202i. In some examples, autonomous vehicle compute 202f includes a device such as a client device, a mobile device (e.g., a cellular telephone, a tablet, and/or the like) a server (e.g., a computing device including one or more central processing units, graphical processing units, and/or the like), and/or the like. In some embodiments autonomous vehicle compute 202f is configured to be in communication with an autonomous vehicle system (e.g., an autonomous vehicle system that is the same as or similar to remote AV system 114 of FIG. 1), a fleet management system (e.g., a fleet management system that is the same as or similar to fleet management system 116 of FIG. 1), a V2I device (e.g., a V2I device that is the same as or similar to V2I device 110 of FIG. 1), and/or a V2I system (e.g., a V2I system that is the same as or similar to V2I system 118 of FIG. 1).

Safety controller 202g includes at least one device configured to be in communication with cameras 202a, LiDAR sensors 202b, radar sensors 202c, microphones 202d, communication device 202e, autonomous vehicle computer 202f, DBW system 202h, and/or ultrasound sensors 202i. In some examples, safety controller 202g includes one or more controllers (electrical controllers, electromechanical controllers, and/or the like) that are configured to generate and/or transmit control signals to operate one or more devices of vehicle 200 (e.g., powertrain control system 204, steering control system 206, brake system 208, and/or the like). In some embodiments, safety controller 202g is configured to generate control signals that take precedence over (e.g., overrides) control signals generated and/or transmitted by autonomous vehicle compute 202f.

DBW system 202h includes at least one device configured to be in communication with communication device 202e and/or autonomous vehicle compute 202f. In some examples, DBW system 202h includes one or more controllers (e.g., electrical controllers, electromechanical controllers, and/or the like) that are configured to generate and/or transmit control signals to operate one or more devices of vehicle 200 (e.g., powertrain control system 204, steering control system 206, brake system 208, and/or the like). Additionally, or alternatively, the one or more controllers of DBW system 202h are configured to generate and/or transmit control signals to operate at least one different device (e.g., a turn signal, headlights, door locks, windshield wipers, and/or the like) of vehicle 200.

Powertrain control system 204 includes at least one device configured to be in communication with DBW system 202h. In some examples, powertrain control system 204 includes at least one controller, actuator, and/or the like. In some embodiments, powertrain control system 204 receives control signals from DBW system 202h and powertrain control system 204 causes vehicle 200 to start moving forward, stop moving forward, start moving backward, stop moving backward, accelerate in a direction, decelerate in a direction, perform a left turn, perform a right turn, and/or the like. In an example, powertrain control system 204 causes the energy (e.g., fuel, electricity, and/or the like) provided to a motor of the vehicle to increase, remain the same, or decrease, thereby causing at least one wheel of vehicle 200 to rotate or not rotate.

Steering control system 206 includes at least one device configured to rotate one or more wheels of vehicle 200. In some examples, steering control system 206 includes at least one controller, actuator, and/or the like. In some embodiments, steering control system 206 causes the front two wheels and/or the rear two wheels of vehicle 200 to rotate to the left or right to cause vehicle 200 to turn to the left or right.

Brake system 208 includes at least one device configured to actuate one or more brakes to cause vehicle 200 to reduce speed and/or remain stationary. In some examples, brake system 208 includes at least one controller and/or actuator that is configured to cause one or more calipers associated with one or more wheels of vehicle 200 to close on a corresponding rotor of vehicle 200. Additionally, or alternatively, in some examples brake system 208 includes an automatic emergency braking (AEB) system, a regenerative braking system, and/or the like.

In some embodiments, vehicle 200 includes at least one platform sensor (not explicitly illustrated) that measures or infers properties of a state or a condition of vehicle 200. In some examples, vehicle 200 includes platform sensors such as a global positioning system (GPS) receiver, an inertial measurement unit (IMU), a wheel speed sensor, a wheel brake pressure sensor, a wheel torque sensor, an engine torque sensor, a steering angle sensor, and/or the like.

Further, the accessibility system 210 includes at least one device configured to detect the location of a user (and/or one or more body parts of the user), and to generate notifications that assist the user in interacting with the vehicle 200. As an example, the accessibility system 210 can detect the location of a user's hand based on sensor data obtained by one or more of the cameras 202a, LiDAR sensors 202b, radar sensors 202c, microphones 202d, and/or ultrasound sensors 202i. Further, the accessibility system 210 can determine the location of the user's hand relative to one or more components of the vehicle 200 and/or other objects on or within the vehicle with which the user can physically interact (e.g., a door handle, a control mechanism, an object left on or within the vehicle, etc.). Further, the accessibility system 210 can generate notifications to aid in the user in guiding her hand towards one or more of the components or objects.

In some embodiments, the accessibility system 210 can be implemented, at least in part, as one or more components of the autonomous system 202. In some embodiments, the accessibility system 210 can be implemented, at least in part, as one or more components or devices that are separate and distinct from the autonomous system 202.

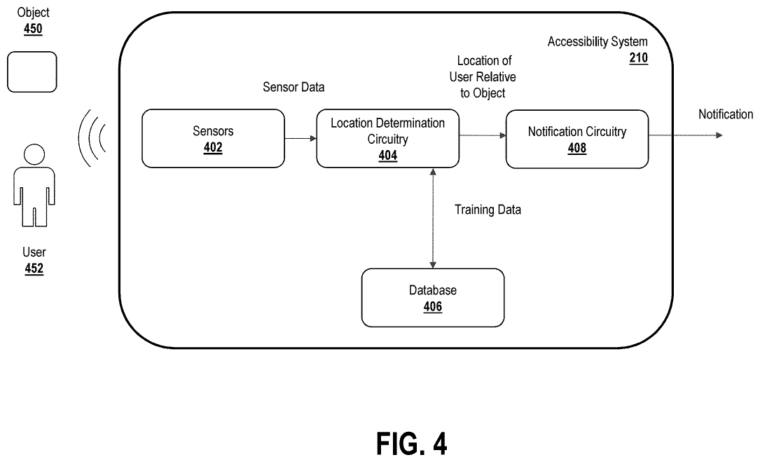

Further details regarding the accessibility system 210 are described, for example, with reference to FIGS. 4-8.

Referring now to FIG. 3, illustrated is a schematic diagram of a device 300. In some embodiments, device 300 corresponds to at least one device of vehicles 102 (e.g., at least one device of a system of vehicles 102) and/or vehicle 200, at least one device of remote AV system 114, fleet management system 116, V2I system 118, and/or one or more devices of network 112 (e.g., one or more devices of a system of network 112). In some embodiments, one or more devices of vehicles 102 and/or 202 (e.g., one or more devices of a system of vehicles 102 and 202, such as the autonomous system 202, the accessibility system 210, etc.), remote AV system 114, fleet management system 116, V2I system 118, and/or one or more devices of network 112 (e.g., one or more devices of a system of network 112) include at least one device 300 and/or at least one component of device 300.

As shown in FIG. 3, device 300 includes bus 302, processor 304, memory 306, storage component 308, input interface 310, output interface 312, and communication interface 314.

Bus 302 includes a component that permits communication among the components of device 300. In some embodiments, processor 304 is implemented in hardware, software, or a combination of hardware and software. In some examples, processor 304 includes a processor (e.g., a central processing unit (CPU), a graphics processing unit (GPU), an accelerated processing unit (APU), and/or the like), a microphone, a digital signal processor (DSP), and/or any processing component (e.g., a field-programmable gate array (FPGA), an application specific integrated circuit (ASIC), and/or the like) that can be programmed to perform at least one function. Memory 306 includes random access memory (RAM), read-only memory (ROM), and/or another type of dynamic and/or static storage device (e.g., flash memory, magnetic memory, optical memory, and/or the like) that stores data and/or instructions for use by processor 304.

Storage component 308 stores data and/or software related to the operation and use of device 300. In some examples, storage component 308 includes a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, a CD-ROM, RAM, PROM, EPROM, FLASH-EPROM, NV-RAM, and/or another type of computer readable medium, along with a corresponding drive.

Input interface 310 includes a component that permits device 300 to receive information, such as via user input (e.g., a touchscreen display, a keyboard, a keypad, a mouse, a button, a switch, a microphone, a camera, and/or the like). Additionally or alternatively, in some embodiments input interface 310 includes a sensor that senses information (e.g., a global positioning system (GPS) receiver, an accelerometer, a gyroscope, an actuator, and/or the like). Output interface 312 includes a component that provides output information from device 300 (e.g., a display, a speaker, one or more light-emitting diodes (LEDs), and/or the like).

In some embodiments, communication interface 314 includes a transceiver-like component (e.g., a transceiver, a separate receiver and transmitter, and/or the like) that permits device 300 to communicate with other devices via a wired connection, a wireless connection, or a combination of wired and wireless connections. In some examples, communication interface 314 permits device 300 to receive information from another device and/or provide information to another device. In some examples, communication interface 314 includes an Ethernet interface, an optical interface, a coaxial interface, an infrared interface, a radio frequency (RF) interface, a universal serial bus (USB) interface, a WiFi® interface, a cellular network interface, and/or the like.

In some embodiments, device 300 performs one or more processes described herein. Device 300 performs these processes based on processor 304 executing software instructions stored by a computer-readable medium, such as memory 305 and/or storage component 308. A computer-readable medium (e.g., a non-transitory computer readable medium) is defined herein as a non-transitory memory device. A non-transitory memory device includes memory space located inside a single physical storage device or memory space spread across multiple physical storage devices.

In some embodiments, software instructions are read into memory 306 and/or storage component 308 from another computer-readable medium or from another device via communication interface 314. When executed, software instructions stored in memory 306 and/or storage component 308 cause processor 304 to perform one or more processes described herein. Additionally or alternatively, hardwired circuitry is used in place of or in combination with software instructions to perform one or more processes described herein. Thus, embodiments described herein are not limited to any specific combination of hardware circuitry and software unless explicitly stated otherwise.

Memory 306 and/or storage component 308 includes data storage or at least one data structure (e.g., a database and/or the like). Device 300 is capable of receiving information from, storing information in, communicating information to, or searching information stored in the data storage or the at least one data structure in memory 306 or storage component 308. In some examples, the information includes network data, input data, output data, or any combination thereof.

In some embodiments, device 300 is configured to execute software instructions that are either stored in memory 306 and/or in the memory of another device (e.g., another device that is the same as or similar to device 300). As used herein, the term “module” refers to at least one instruction stored in memory 306 and/or in the memory of another device that, when executed by processor 304 and/or by a processor of another device (e.g., another device that is the same as or similar to device 300) cause device 300 (e.g., at least one component of device 300) to perform one or more processes described herein. In some embodiments, a module is implemented in software, firmware, hardware, and/or the like.

The number and arrangement of components illustrated in FIG. 3 are provided as an example. In some embodiments, device 300 can include additional components, fewer components, different components, or differently arranged components than those illustrated in FIG. 3. Additionally or alternatively, a set of components (e.g., one or more components) of device 300 can perform one or more functions described as being performed by another component or another set of components of device 300.

Example Accessibility Systems

FIG. 4 shows aspects of the accessibility system 210 in greater detail. The accessibility system 210 includes one or more sensors 402, location determination circuitry 404, a database 406, and notification circuitry 408.

In general, the accessibility system 210 is configure to detect the location of a user 452, and determine the location of the user 452 relative to a particular goal (e.g., an object 450). Further, the accessibility system 210 is configured to generate notifications to the user 452 to guide the user 452 towards the goal.

In some implementations, the user 452 and/or the object 450 can located in an exterior environment of the vehicle 200. In some implementations, the user 452 and/or the object 450 can located in an interior of the vehicle 200 (e.g., a passenger compartment of the vehicle 200).

As an example, accessibility system 210 can determine the location of a user's hand, and determine the location of the user's hand relative to an exterior door handle of the vehicle 200 (e.g., a distance and/or direction between the user's hand and the door handle). Further, the accessibility system 210 can generate notifications (e.g., auditory alerts, visual alerts, haptic alerts, etc.) that guide the user's hand towards the door handle, such that the user can grasp and manipulate the door handle.

As another example, accessibility system 210 can determine the location of a user's hand, and determine the location of the user's hand relative to a control mechanism within the vehicle 200 (e.g., a wheel, a button, a lever, etc.). Further, the accessibility system 210 can generate notifications that guide the user's hand towards the control mechanism, such that the user can physically interact with the control mechanism.

As another example, accessibility system 210 can determine the location of a user's hand, and determine the location of the user's hand relative to an object left within the vehicle 200 (e.g., a personal item). Further, the accessibility system 210 can generate notifications that guide the user's hand towards the object, such that the user can locate and retrieve the object.

Referring to FIG. 4, the accessibility system 210 gathers sensor measurements using one or more of the sensors 402. In some implementations, the sensors 402 can include one or more cameras (e.g., for gathering still images and/or videos), LiDAR sensors (e.g., for gathering LiDAR data), radar sensors (e.g., for gathering radar data), microphones (e.g., for gathering sound recordings), and/or ultrasound sensors (e.g., for gathering ultrasonic sensor data). For example, the sensors 402 can include one or more of the cameras 202a, LiDAR sensors 202b, radar sensors 202c, microphones 202d, and/or ultrasound sensors 202i described with reference to FIG. 2.

In some implementations, at least some of the sensors 402 can be configured to gather sensor data regarding an exterior environment of the vehicle 200 and/or one or more objects in that exterior environment. As an example, the sensors 402 can be directed towards an exterior of the vehicle 200, and gather sensor measurements representing a user 452 and/or an object 450 located in the exterior environment of the vehicle 200.

In some implementations, at least some of the sensors 402 can be configured to gather sensor data regarding an interior of the vehicle 200 and/or one or more objects in that interior. As an example, the sensors 402 can be directed towards an interior of the vehicle 200 (e.g., a passenger cabin of the vehicle 200, a cargo compartment of the vehicle, etc.), and gather sensor measurements representing a user 452 and/or an object 450 located in the interior of the vehicle 200.

In some implementations, at least some of the sensors 402 can be configured to provide sensor data to the accessibility system 210, as well as to the autonomous system 202 of the vehicle 200 (e.g., to facilitate the performance of autonomous operations, such as autonomous navigation) and/or any other system of the vehicle 200. This can be beneficial, for example, in reducing the complexity of the vehicle 200 (e.g., by reducing the number of components in the vehicle 200 that are dedicated for a single purpose). However, in some implementations, at least some of the sensors 402 can be configured to provide sensor data exclusively to the accessibility system 210 (e.g., to facilitate performance of the accessibility operations described herein).

The location determination circuitry 404 receives sensor data from the sensors 402, and determines the location of the user 452 and/or the object 450.

In some implementations, the location determination circuitry 404 can detect one or more specific body parts of the user 452, and determine the location of each of those body parts. As an example, based on the sensor data, the location determination circuitry 404 can detect a user's hand, arm, finger, foot, leg, head, torso, and/or any other body part. Further, based on the sensor data, the location determination circuitry 404 can determine the location of each of the body parts.

Further, the location determination circuitry 404 can detect one or more objects 450, and determine the location of each of those objects 450.

In some implementations, the location determination circuitry 404 can determine the absolute location of the user 452 (and/or the user's body parts) and/or the object 450, such as a location expressed according to geographical coordinates (e.g., latitude, longitude, altitude, etc.).

In some implementations, the location determination circuitry 404 can determine the location of the user 452 (and/or the user's body parts) and/or the object 450 relative to a frame of reference of the vehicle 200 (e.g., a set of x-, y-, and z-coordinates having the vehicle of a frame of reference). In some implementations, the object 450 may be secured to a known location on the vehicle, and the location determination circuitry 404 can determine location of the object 450 without obtaining sensor data specifically regarding the object 450.

In some implementations, the location determination circuitry 404 can determine the location of the user 452 (and/or one or more of the user's body parts) relative to the object 450. For example, the location determination circuitry 404 can determine the distance between the location of the user 452 (and/or one or more of the user's body parts) and a location of the object 450. As another example, the location determination circuitry 404 can determine the relative direction from the user 452 (and/or one or more of the user's body parts) to the object 450 (e.g., a direction vector and/or an angular value).

In some implementations, the location determination circuitry 404 can determine an identity of the user 452 based on the sensor data obtained by the sensors 402. For example, the location determination circuitry 404 can determine that the user 452 is a particular user from among a set of candidate users.

Further, the location determination circuitry 404 can determine, for each of the user's body parts, the identity of that body part. For example, based on the sensor data obtained by the sensors 402, the location determination circuitry 404 can distinguish different body parts of the user from one another, and identify the locations of one or more specific types of body parts (e.g., hands, arms, feet, legs, torso, head, etc.).

In some implementations, the location determination circuitry 404 can determine an identity of the object 450 based on the sensor data obtained by the sensors 402. For example, the location determination circuitry 404 can determine that the object 450 is of a particular object type. Example object types include a door handle, a steering wheel, a button, a lever, a switch, a latch, or any of control mechanism of a vehicle 200. Further example object types include a seat belt (e.g., a buckle, tongue, strap, etc. thereof), a backpack, a purse, a handbag, a wallet, a suitcase, a briefcase, luggage, an article of clothing (e.g., a coat, a shirt, pants, a hat, etc.), an electronic device (e.g., a computer, a smart phone, a tablet, headphones, earbuds, etc.), glasses, sports equipment (e.g., a ball, a bat, a racket, a golf club, a helmet, etc.), a tool (e.g., a hammer, a wrench, a screwdriver, etc.), jewelry (e.g., a ring, a watch, earrings, a necklace, etc.), and/or any other type of object.

In some implementations, the location determination circuitry 404 can make at least some of the determinations described herein based on one or more machine learning models. For example, a machine learning model can be trained to receive input data (e.g., data received from the sensors 402 and/or sensors similar to the sensors 402), and, based on the input data, generate output data associated with one or more predictions regarding the locations and/or identities of the user 452, the body parts of the users 452, and/or the object 450.

As an example, a machine learning model can be trained using training data regarding one or more users or objects that were previously detected by a vehicle 200 or another vehicle (e.g., training data stored in the database 406). In some implementations, these users or objects can include users or objects that were previously detected and/or identified by the accessibility system 210. These users or objects also can include users or objects that were previously detected and/or identified by another system (e.g., another accessibility system 210).

For each of the users or objects, the training data can include input information similar to that described with reference to FIG. 4. For example, the training data can include data obtained by one or more sensors (e.g., cameras, LiDAR sensors, radar sensors, microphones, ultrasonic sensor data, etc.) while the user or object was located within or in a vicinity of a vehicle.

Further, for each of the users or objects, the training data can include data representing a location of that user or object at the time that the sensor measurements were obtained. For example, the training data can indicate a general area in which a particular user or object was located at the time that the sensor measurements were obtained. As another example, the training data can indicate a set of spatial coordinates representing the location of a particular user or object at the time that the sensor measurements were obtained. As another example, the training data can indicate a set of spatial coordinates representing the location of a particular user or object relative to another user and/or object, at the time that the sensor measurements were obtained.

Further, for each of the users, the training data can include data representing an identity of that user, and the identity of each of the user's body parts represented by the sensor data. For example, the training data can indicate the name of a user. As another example, the training data can include annotations indicating which of the user's body parts are represented by each portion of sensor data, and the identity of each of those body parts. For instance, the training data can include an image of a user, an indication of the user's name, and annotations indicating the locations of one or more of the user's body parts that are visible in the image.

Further, for each of the objects, the training data can include data representing an identity or type of that object. For example, the training data can indicate whether a particular object is a door handle, control mechanism, backpack, purse, handbag, wallet, suitcase, briefcase, luggage, article of clothing, electronic device, glasses, sports equipment, tool, jewelry, and/or any other type of object. For instance, the training data can include an image of an object, an indication of the object's identity (e.g., object type), and annotations indicating the location of the object in the image.

Based on the training data, the machine learning model can be trained to identify correlations, relationships, and/or trends between (i) the input data, (ii) the location of a user (and/or the user's body parts), and/or (iii) the identity of that user (e.g., the user's body parts). Further, based on the training data, the machine learning model can be trained to identify correlations, relationships, and/or trends between (i) the input data, (ii) the location of an object, and/or (iii) the identity of that object.

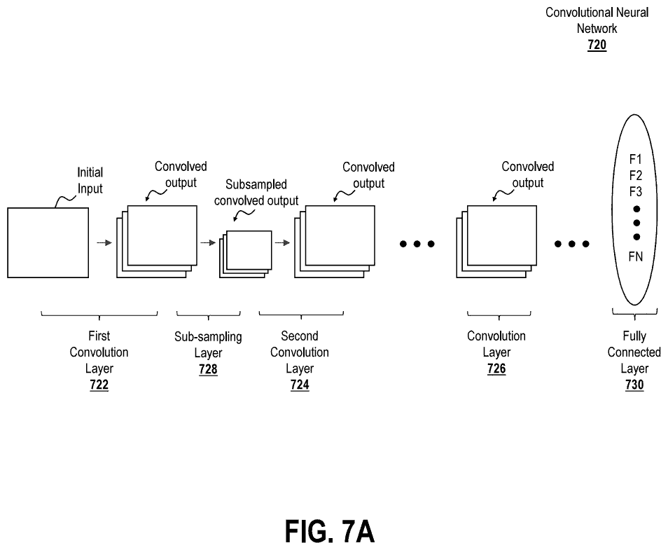

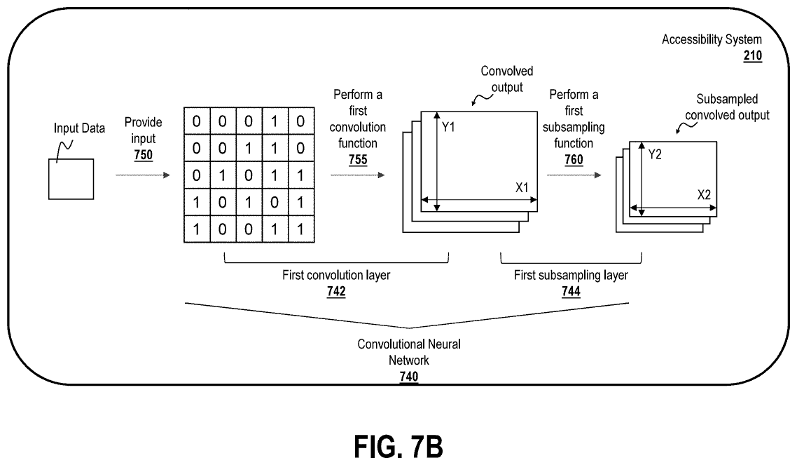

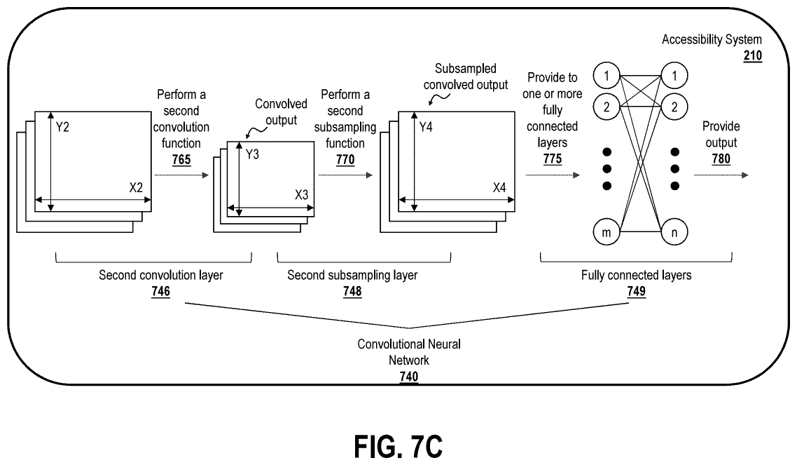

Example machine learning models are described in further detail with reference to FIGS. 7A-7C.

In some embodiments, the accessibility system 210 can be configured to store information regarding the user 452 and/or the object 450 (e.g., in the database 406) for future retrieval and/or processing. As an example, the location determination circuitry 404 can transmit information regarding the user 452, such as the determined location of the user 452 and/or the determined identify of the user 452 to the database 406 for storage. As another example, the location determination circuitry 404 can transmit information regarding the object 450, such as the determined location of the object 450 and/or the determined identity of that object 450 to the database 406 for storage. As another example, the location determination circuitry 404 can transmit at least some of the sensor information obtained regarding the user 452 and/or object 450 (e.g., images, videos, LiDAR data, radar data, sound recordings, ultrasonic sensor data, etc.) to the database 406 for storage.

Further, the accessibility system 210 can be configured to generate notifications to the user 452 (e.g., to guide the user 452 towards a goal, such as the object 450). As an example, the location determination circuitry 404 can determine the location of the user 452 relative to the location of the object 450 (e.g., a relative distance between them and/or a relative direction between them). Further, based on this information, the notification circuitry 408 can generate one or more notifications to the user 452, such that that the user can locate and interact with the object 450.

In some implementations, a notification can include audio content (e.g., one or more beeps, tones, spoken words, etc.), visual content (e.g., videos, icons, illuminated and/or flashing lights, etc.), and/or haptic feedback (e.g., a vibration or pulse). In some implementations, a notification can be generated, at least in part, using an electronic device (e.g., a smart phone, a tablet computer, and/or a wearable computer carried or worn by a user). In some implementations, a notification can be generated, least in part, using the vehicle 200 itself (e.g., using one or more audio speakers, horns, display devices, status lights, etc. of the vehicle 200).

In some implementations, the accessibility system 210 can be configured to generate notifications to the user 452 can be configured to generate notifications continuously or periodically until a particular stop condition is satisfied. As an example, the accessibility system 210 can be configured to generate notifications to the user 452 until the user comes into contact with the object, when the user comes within a particular threshold distance from the object, and/or when the user manually instructs the accessibility system 210 to cease providing further notifications.

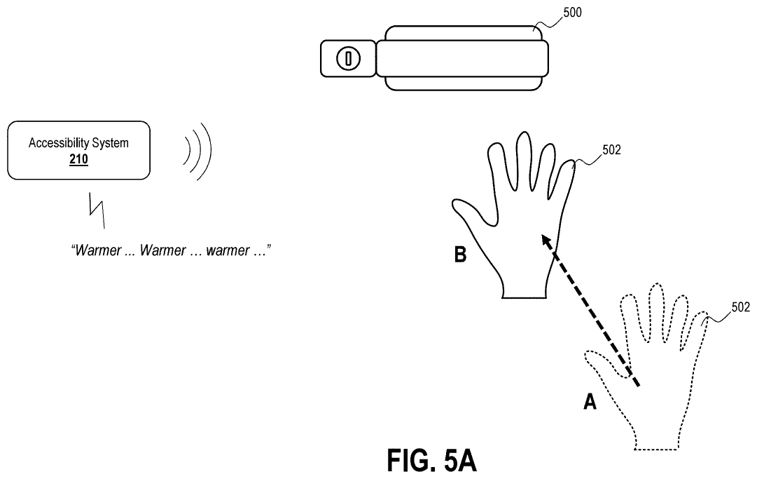

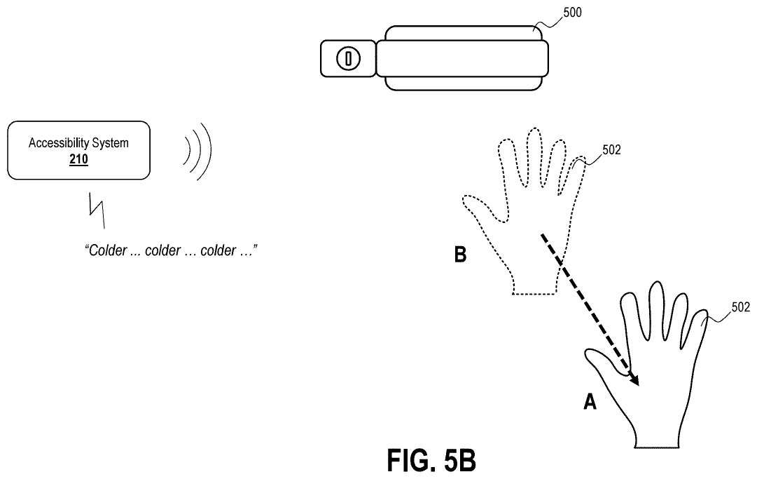

FIGS. 5A and 5B show an example operation of the accessibility system 210. In this example, a user is exterior to a vehicle, and is attempting to gain access to the vehicle (e.g., by grasping and manipulating a door handle 500 with her hand 502). As shown in FIG. 5A, the accessibility system 210 can determine the location of the user's hand 502 relative to the location of the door handle 500. As the user brings her hand 502 closer to the door handle 500 (e.g., from a position A to a position B), the accessibility system 210 can generate a notification indicating that the user's hand 502 is moving toward the door handle 500.

As an example, the accessibility system 210 can generate an audio message as the user's hand 502 is brought closer to the door handle 500 (e.g., “warmer . . . warmer . . . warmer . . . ”). As another example, the accessibility system 210 can generate an audio tone that changes in volume (e.g., increases in volume) as the user's hand 502 is brought closer to the door handle 500. As another example, the accessibility system 210 can generate an audio tone that changes in pitch or frequency (e.g., increases in pitch or frequency) as the user's hand 502 is brought closer to the door handle 500. As another example, the accessibility system 210 can generate a video and/or illuminate a status light in a particular pattern as the user's hand 502 is brought closer to the door handle 500. As another example, the accessibility system 210 can generate a haptic feedback having a particular pattern as the user's hand 502 is brought closer to the door handle 500.

As shown in FIG. 5B, as the user moves her hand 502 further from the door handle 500 (e.g., from the position B to the position A), the accessibility system 210 can generate a notification indicating that the user's hand 502 is moving away the door handle 500.

As an example, the accessibility system 210 can generate an audio message as the user's hand 502 is moved farther from the door handle 500 (e.g., “colder . . . colder . . . colder . . . ”). As another example, the accessibility system 210 can generate an audio tone that changes in volume (e.g., decreases in volume) as the user's hand 502 is moved farther from the door handle 500. As another example, the accessibility system 210 can generate an audio tone that changes in pitch or frequency (e.g., decreases in pitch or frequency) as the user's hand 502 is moved farther from the door handle 500. As another example, the accessibility system 210 can generate a video and/or illuminate a status light in a different particular pattern as the user's hand 502 is moved farther from the door handle 500. As another example, the accessibility system 210 can generate a haptic feedback having a different particular pattern as the user's hand 502 is moved farther from the door handle 500.

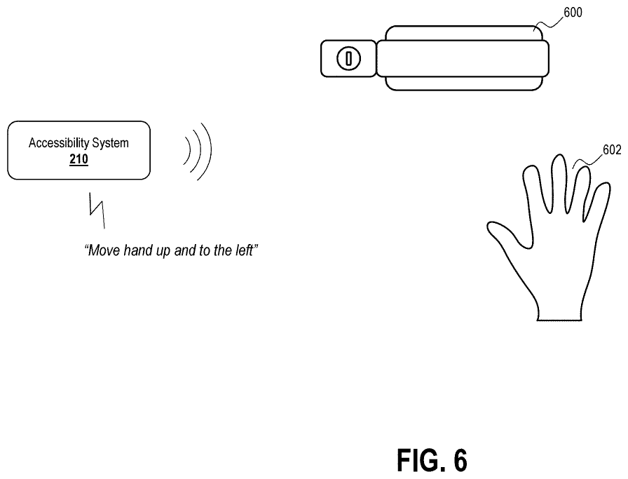

FIG. 6 shows another example operation of the accessibility system 210. In this example, a user is exterior to a vehicle, and is attempting to gain access to the vehicle (e.g., by grasping and manipulating a door handle 600 with her hand 602). As shown in FIG. 6, the accessibility system 210 can determine the location of the user's hand 602 relative to the location of the door handle 600. Further, the accessibility system 210 can generate a notification indicating a relative direction from the user's hand 602 to the door handle 600 (e.g., such that that user can move her hand 602 towards the door handle 600).

As an example, the accessibility system 210 can generate an audio message guiding the user's hand 602 towards the door handle 600 (e.g., “move hand up and to the left”). As an example, the accessibility system 210 can generate a sound near the door handle 600 (e.g., using a speaker that is near the door handle 600). As another example, the accessibility system 210 can simulate the emission of sound from the door handle 500 (e.g., by generating directional sound using two or more speakers concurrently, such that sound appears to be emitted from the location of the door handle). As another example, the accessibility system 210 can generate a video and/or illuminate a status light in a different particular pattern to indicate a suggest direction of movement for the user's hand 602. As another example, the accessibility system 210 can generate a haptic feedback having a different particular pattern to indicate a suggested direction of movement for the user's hand 602.

Although FIGS. 5A, 5B, and 6 show example use cases in which the accessibility system 210 assists a user in interacting with a door handle, in practice, the accessibility system 210 can also be used to assist the user in interacting with any other object, either on an exterior of the vehicle 200 and/or in an interior of the vehicle 200.

As an example, the accessibility system 210 can be configured to identify the location of a user's hand, and guide the user's hand towards a door handle on an interior of the vehicle (e.g., the assist the user in exiting the vehicle).

As another example, the accessibility system 210 can be configured to identify the location of a user's hand, and guide the user's hand towards a control mechanism (e.g., a steering wheel, a button, a lever, a switch, a latch, or any other control mechanism), to assist the user in controlling the operation of the vehicle.

As another example, the accessibility system 210 can be configured to identify the location of a user's hand, and guide the user's hand towards a seat belt (e.g., a buckle, a tongue, and/or a strap of the steal belt) to assist the user in securing herself or others within the vehicle.

As another example, the accessibility system 210 can be configured to identify the location of a user's hand, and guide the user's hand towards an object in the vehicle (e.g., a personal item left in the vehicle), such that the user can locate and retrieve the object.

In some implementations, the accessibility system 210 can also aid a user in selecting a particular door and/or seat of a vehicle, prior to the user entering the vehicle. This can be particularly advantageous in vehicles that are shared among several different users (e.g., an autonomous vehicle used in a ride-sharing service), in which a user may be assigned a vehicle that is already occupied by one or more other users.

As an example, the accessibility system 210 can be configured to obtain sensor data regarding the interior of a vehicle, and determine whether each seat of the vehicle is currently occupied. For instance, at least some of the sensors 402 can be directed towards one or more seats in the passenger compartment of the vehicle, and generate sensor data indicating whether the seat is currently occupied by a passenger and/or an object (e.g., a bag, box, or other object that may obstruct the seat). Based on the sensor data, the accessibility system 210 can select an empty seat for the user, and direct the user towards the selected seat and/or the vehicle door that is proximate to the selected seat.

In some implementations, the accessibility system 210 can generate notifications that guide the user to the selected seat and/or door, in a similar manner as described above. For example, the accessibility system 210 can generate one or more notifications that include audio content (e.g., one or more beeps, tones, spoken words, etc.), visual content (e.g., videos, icons, illuminated and/or flashing lights, etc.), and/or haptic feedback (e.g., a vibration or pulse). In some implementations, a notification can be generated, at least in part, using an electronic device (e.g., a smart phone, a tablet computer, and/or a wearable computer carried or worn by a user). In some implementations, a notification can be generated, least in part, using the vehicle 200 itself (e.g., using one or more audio speakers, horns, display devices, status lights, etc. of the vehicle 200).

Further, the accessibility system 210 can generate notifications to guide the user in entering the vehicle, as described above. For example, the accessibility system 210 can generate notifications to guide the user's hand to a door handle, such that the user can manipulate the door handle, open the door, and enter the vehicle.

In some implementations, the accessibility system 210 can be automatically activated to provide guidance to a user. For example, if the vehicle 200 is a vehicle that is shared among several different users (e.g., a vehicle that is used in a ride-sharing service), the accessibility system 210 can determine whether the user to which the vehicle is currently assigned (or will be assigned) has requested guidance from an accessibility system and/or suffers from a disability (e.g., is visually impaired). If so, the accessibility system 210 can automatically provide guidance to aid in the user's entering, operating, and/or exiting the vehicle. In some implementations, information regarding a user can be determined based on user profile information stored by a computer system of the vehicle 200 and/or the ride-sharing service.

As another example, the accessibility system 210 can predict whether a user would like to receive guidance. For instance, the accessibility system 210 can determine one or more actions being performed by the user (e.g., based on sensor data), and determine whether those actions are indicative of the user requiring assistance. As an example, a user may require assistance if she is moving her hand along a door of the vehicle, but has not located a door handle of the vehicle. In some implementations, the accessibility system 210 can make a prediction, at least in part, using a machine learning model.

In some implementations, a user can selectively request that the accessibility system 210 provide guidance to the user. For example, the user can input a command instructing the accessibility system 210 to provide guidance to the user. In some implementation, the command can include a spoken command, a physical gesture, physical interaction (e.g., pressing a button, touching a touch sensitive display device, etc.), or any other action. In response, the accessibility system 210 can provide guidance to aid in the user's entering, operating, and/or exiting the vehicle.

At least some of the techniques describe herein can be implemented using one or more machine learning models. As an example, FIG. 7A shows a diagram of an implementation of a machine learning model. More specifically, illustrated is a diagram of an implementation of a convolutional neural network (CNN) 720. For purposes of illustration, the following description of CNN 720 will be with respect to an implementation of CNN 720 by the accessibility system 210. However, it will be understood that in some examples CNN 720 (e.g., one or more components of CNN 720) is implemented by other systems different from, or in addition to, the accessibility system 210, such as the autonomous vehicle compute 202f. While CNN 720 includes certain features as described herein, these features are provided for the purpose of illustration and are not intended to limit the present disclosure.

CNN 720 includes a plurality of convolution layers including first convolution layer 722, second convolution layer 724, and convolution layer 726. In some embodiments, CNN 720 includes sub-sampling layer 728 (sometimes referred to as a pooling layer). In some embodiments, sub-sampling layer 728 and/or other subsampling layers have a dimension (i.e., an amount of nodes) that is less than a dimension of an upstream system. By virtue of sub-sampling layer 728 having a dimension that is less than a dimension of an upstream layer, CNN 720 consolidates the amount of data associated with the initial input and/or the output of an upstream layer to thereby decrease the amount of computations necessary for CNN 720 to perform downstream convolution operations. Additionally, or alternatively, by virtue of sub-sampling layer 728 being associated with (e.g., configured to perform) at least one subsampling function (as described below with respect to FIGS. 7B and 7C), CNN 720 consolidates the amount of data associated with the initial input.

The accessibility system 210 performs convolution operations based on the accessibility system 210 providing respective inputs and/or outputs associated with each of first convolution layer 722, second convolution layer 724, and convolution layer 726 to generate respective outputs. In some examples, the accessibility system 210 implements CNN 720 based on the accessibility system 210 providing data as input to first convolution layer 722, second convolution layer 724, and convolution layer 726. In such an example, the accessibility system 210 provides the data as input to first convolution layer 722, second convolution layer 724, and convolution layer 726 based on the accessibility system 210 receiving data from one or more different systems (e.g., the sensors 402, the database 406, etc.). A detailed description of convolution operations is included below with respect to FIG. 7B.

In some embodiments, the accessibility system 210 provides data associated with an input (referred to as an initial input) to first convolution layer 722 and the accessibility system 210 generates data associated with an output using first convolution layer 722. In some embodiments, the accessibility system 210 provides an output generated by a convolution layer as input to a different convolution layer. For example, the accessibility system 210 provides the output of first convolution layer 722 as input to sub-sampling layer 728, second convolution layer 724, and/or convolution layer 726. In such an example, first convolution layer 722 is referred to as an upstream layer and sub-sampling layer 728, second convolution layer 724, and/or convolution layer 726 are referred to as downstream layers. Similarly, in some embodiments the accessibility system 210 provides the output of sub-sampling layer 728 to second convolution layer 724 and/or convolution layer 726 and, in this example, sub-sampling layer 728 would be referred to as an upstream layer and second convolution layer 724 and/or convolution layer 726 would be referred to as downstream layers.

In some embodiments, the accessibility system 210 processes the data associated with the input provided to CNN 720 before the accessibility system 210 provides the input to CNN 720. For example, the accessibility system 210 processes the data associated with the input provided to CNN 720 based on the accessibility system 210 and normalizing sensor data (e.g., audio data, image data, video data, and/or the like).

In some embodiments, CNN 720 generates an output based on the accessibility system 210 performing convolution operations associated with each convolution layer. In some examples, CNN 720 generates an output based on the accessibility system 210 performing convolution operations associated with each convolution layer and an initial input. In some embodiments, the accessibility system 210 generates the output and provides the output as fully connected layer 730. In some examples, the accessibility system 210 provides the output of convolution layer 726 as fully connected layer 730, where fully connected layer 730 includes data associated with a plurality of feature values referred to as F1, F2 . . . FN. In this example, the output of convolution layer 726 includes data associated with a plurality of output feature values that represent a prediction.

In some embodiments, the accessibility system 210 identifies a prediction from among a plurality of predictions based on the accessibility system 210 identifying a feature value that is associated with the highest likelihood of being the correct prediction from among the plurality of predictions. For example, where fully connected layer 730 includes feature values F1, F2, . . . FN, and F1 is the greatest feature value, the accessibility system 210 identifies the prediction associated with F1 as being the correct prediction from among the plurality of predictions. In some embodiments, the accessibility system 210 trains CNN 720 to generate the prediction. In some examples, the accessibility system 210 trains CNN 720 to generate the prediction based on the accessibility system 210 providing training data associated with the prediction to CNN 720.

A prediction can include, for example, a predicted location of a user and/or the user's body parts, either within a vehicle 200 or in an exterior environment of a vehicle 200. As another example, a prediction can include the identity of the user and/or the user's body parts. As another example, a prediction can include a predicted location of an object, either within a vehicle 200 or in an exterior environment of a vehicle 200. As another example, a prediction can include the identity of the object.

Referring now to FIGS. 7B and 7C, illustrated is a diagram of example operation of CNN 740 by the accessibility system 210. In some embodiments, CNN 740 (e.g., one or more components of CNN 740) is the same as, or similar to, CNN 720 (e.g., one or more components of CNN 720) (see FIG. 7A).

At step 750, the accessibility system 210 provides data as input to CNN 740 (step 750). For example, the accessibility system 210 can provide data obtained by one or more of the sensors 402. As another example, the object detection system 210 can provide data received from the database 406.

At step 755, CNN 740 performs a first convolution function. For example, CNN 740 performs the first convolution function based on CNN 740 providing the values representing the input data as input to one or more neurons (not explicitly illustrated) included in first convolution layer 742. As an example, the values representing an image or video can correspond to values representing a region of the image or video (sometimes referred to as a receptive field). As another example, the values representing an audio signal can correspond to values representing a portion or the audio signal (e.g., a particular temporal portion and/or a particular spectral portion). As another example, the values representing some other sensor measurement can correspond to values representing a portion of that sensor measurement (e.g., a particular temporal portion and/or a particular spectral portion).

In some embodiments, each neuron is associated with a filter (not explicitly illustrated). A filter (sometimes referred to as a kernel) is representable as an array of values that corresponds in size to the values provided as input to the neuron. In one example, a filter may be configured to identify edges in an image (e.g., horizontal lines, vertical lines, straight lines, and/or the like). In successive convolution layers, the filters associated with neurons may be configured to identify successively more complex patterns in the image (e.g., arcs, objects, and/or the like). In another example, a filter may be configured to identify spectral portions of an audio signal (e.g., portions of an audio signal corresponding to particular frequencies and/or frequency ranges). In successive convolution layers, the filters associated with neurons may be configured to identify successively more complex patterns in the audio signal (e.g., patterns indicative of a location of a source of the audio, an identity or type of the source of the audio, etc.).

In some embodiments, CNN 740 performs the first convolution function based on CNN 740 multiplying the values provided as input to each of the one or more neurons included in first convolution layer 742 with the values of the filter that corresponds to each of the one or more neurons. For example, CNN 740 can multiply the values provided as input to each of the one or more neurons included in first convolution layer 742 with the values of the filter that corresponds to each of the one or more neurons to generate a single value or an array of values as an output. In some embodiments, the collective output of the neurons of first convolution layer 742 is referred to as a convolved output. In some embodiments, where each neuron has the same filter, the convolved output is referred to as a feature map.

In some embodiments, CNN 740 provides the outputs of each neuron of first convolutional layer 742 to neurons of a downstream layer. For purposes of clarity, an upstream layer can be a layer that transmits data to a different layer (referred to as a downstream layer). For example, CNN 740 can provide the outputs of each neuron of first convolutional layer 742 to corresponding neurons of a subsampling layer. In an example, CNN 740 provides the outputs of each neuron of first convolutional layer 742 to corresponding neurons of first subsampling layer 744. In some embodiments, CNN 740 adds a bias value to the aggregates of all the values provided to each neuron of the downstream layer. For example, CNN 740 adds a bias value to the aggregates of all the values provided to each neuron of first subsampling layer 744. In such an example, CNN 740 determines a final value to provide to each neuron of first subsampling layer 744 based on the aggregates of all the values provided to each neuron and an activation function associated with each neuron of first subsampling layer 744.

At step 760, CNN 740 performs a first subsampling function. For example, CNN 740 can perform a first subsampling function based on CNN 740 providing the values output by first convolution layer 742 to corresponding neurons of first subsampling layer 744. In some embodiments, CNN 740 performs the first subsampling function based on an aggregation function. In an example, CNN 740 performs the first subsampling function based on CNN 740 determining the maximum input among the values provided to a given neuron (referred to as a max pooling function). In another example, CNN 740 performs the first subsampling function based on CNN 740 determining the average input among the values provided to a given neuron (referred to as an average pooling function). In some embodiments, CNN 740 generates an output based on CNN 740 providing the values to each neuron of first subsampling layer 744, the output sometimes referred to as a subsampled convolved output.

At step 765, CNN 740 performs a second convolution function. In some embodiments, CNN 740 performs the second convolution function in a manner similar to how CNN 740 performed the first convolution function, described above. In some embodiments, CNN 740 performs the second convolution function based on CNN 740 providing the values output by first subsampling layer 744 as input to one or more neurons (not explicitly illustrated) included in second convolution layer 746. In some embodiments, each neuron of second convolution layer 746 is associated with a filter, as described above. The filter(s) associated with second convolution layer 746 may be configured to identify more complex patterns than the filter associated with first convolution layer 742, as described above.

In some embodiments, CNN 740 performs the second convolution function based on CNN 740 multiplying the values provided as input to each of the one or more neurons included in second convolution layer 746 with the values of the filter that corresponds to each of the one or more neurons. For example, CNN 740 can multiply the values provided as input to each of the one or more neurons included in second convolution layer 746 with the values of the filter that corresponds to each of the one or more neurons to generate a single value or an array of values as an output.

In some embodiments, CNN 740 provides the outputs of each neuron of second convolutional layer 746 to neurons of a downstream layer. For example, CNN 740 can provide the outputs of each neuron of first convolutional layer 742 to corresponding neurons of a subsampling layer. In an example, CNN 740 provides the outputs of each neuron of first convolutional layer 742 to corresponding neurons of second subsampling layer 748. In some embodiments, CNN 740 adds a bias value to the aggregates of all the values provided to each neuron of the downstream layer. For example, CNN 740 adds a bias value to the aggregates of all the values provided to each neuron of second subsampling layer 748. In such an example, CNN 740 determines a final value to provide to each neuron of second subsampling layer 748 based on the aggregates of all the values provided to each neuron and an activation function associated with each neuron of second subsampling layer 748.

At step 770, CNN 740 performs a second subsampling function. For example, CNN 740 can perform a second subsampling function based on CNN 740 providing the values output by second convolution layer 746 to corresponding neurons of second subsampling layer 748. In some embodiments, CNN 740 performs the second subsampling function based on CNN 740 using an aggregation function. In an example, CNN 740 performs the first subsampling function based on CNN 740 determining the maximum input or an average input among the values provided to a given neuron, as described above. In some embodiments, CNN 740 generates an output based on CNN 740 providing the values to each neuron of second subsampling layer 748.

At step 775, CNN 740 provides the output of each neuron of second subsampling layer 748 to fully connected layers 749. For example, CNN 740 provides the output of each neuron of second subsampling layer 748 to fully connected layers 749 to cause fully connected layers 749 to generate an output. In some embodiments, fully connected layers 749 are configured to generate an output associated with a prediction (sometimes referred to as a classification).

As an example, the output can include a prediction regarding the location of a user and/or one or more of the user's body parts. For instance, the output can indicate a set of geographical coordinates and/or spatial coordinates representing the absolute location of the user or the location of the user relative to a frame of reference (e.g., the vehicle and/or another object).

As another example, the output can include a prediction regarding the identity to the user and/or the user's body parts. For instance, the output can indicate a name of user. Further, the output can indicate which ones of the user's body parts have been detected by the accessibility system 210.

As another example, the output can include a prediction regarding the location of a object. For instance, the output can indicate a set of geographical coordinates and/or spatial coordinates representing the absolute location of the object or the location of the user relative to a frame of reference (e.g., the user, the vehicle, and/or another object).

As an example, the output can include a prediction regarding the identity or type of an object. For example, the output can indicate a whether the object is a door handle, control mechanism, backpack, purse, handbag, wallet, suitcase, briefcase, luggage, article of clothing, electronic device, glasses, sports, tool, jewelry, and/or any other type of object.

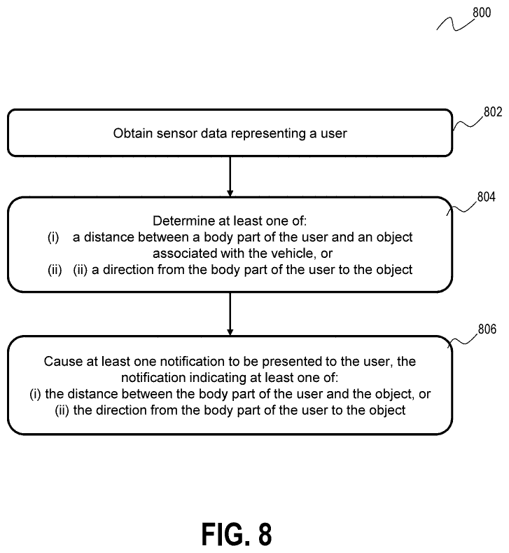

Referring now to FIG. 8, illustrated is a flowchart of a process 800 for assisting a user in interacting with a vehicle. In some embodiments, one or more of the steps described with respect to process 800 are performed (e.g., completely, partially, and/or the like) by the accessibility system 210. Additionally, or alternatively, in some embodiments one or more steps described with respect to process 800 are performed (e.g., completely, partially, and/or the like) by another device or group of devices separate from or including the accessibility system 210, such as a computer system remote from a vehicle (e.g., a server computer and/or a cloud computer system).

With continued reference to FIG. 8, an accessibility system of a vehicle obtains sensor data representing a user (block 802). The sensor data can be obtained from a sensor system of the vehicle, such as a sensor system including one or more cameras, radar sensors, LiDAR sensors, and ultrasound sensors. For instance, the sensor data can include one or more images, videos, radar data, LiDAR data, and/or ultrasonic sensor data.

With continued reference to FIG. 8, the accessibility system determines at least one of (i) a distance between a body part of the user and an object associated with the vehicle, or (ii) a direction from the body part of the user to the object (block 804).

In some implementations, the body part of the user can include a hand of the user, a finger of the user, and/or a foot of the user.

Further, the object can include a door handle of the vehicle, a control mechanism of the vehicle (e.g., a steering wheel, a button, a lever, a switch, a latch, or any other control mechanism), a seat beat of the vehicle (or a component thereof), or an object left within the vehicle (e.g., a personal item).

In some implementations, the accessibility system can continuously determine (i) the distance between the body part of the user and the object and/or (ii) the direction extending from the body part of the user to the object, until a stop condition is satisfied. Further, the accessibility system can continuously cause the at least one notification to be presented to the user, until a stop condition is satisfied. In some implementations, the stop condition can include a determination that the body part of the user is in contact with the object. In some implementations, the stop condition can include a determination that the body part of the user is within a threshold distance from the object.

With continued reference to FIG. 8, the accessibility system causes at least one notification to be presented to the user (block 806). The least one notification indicates at least one of: (i) the distance between the body part of the user and the object, or (ii) the direction from the body part of the user to the object.

In some implementations, a notification can include an audio content (e.g., an auditory signal), haptic feedback, a visual content (e.g., a video, an indicator light, etc.).

In some implementations, a notification can include an auditory signal. Further, a frequency of the auditory signal can vary depending on the distance between the body part of the user and the object (e.g., a higher frequency signal when the user is closer to the object, and a lower frequency signal when the user is farther from the object).

In some implementations, causing the at least one notification to be presented to the user can include causing an audio speaker of the vehicle to present the at least one notification.

In some implementations, causing the at least one notification to be presented to the user can include causing a mobile device associated with the user to present the at least one notification. The mobile device can include at least one of a communications device (e.g., smart phone, tablet computer, etc.) or a wearable device (e.g., smart watch).

Although the example embodiments described herein pertain to assisting a user in interacting with a vehicle, other embodiments can be used to assist a user in interacting with systems or devices other than vehicles.

As an example, one or more of the systems, methods, and/or computer program products described herein can be configured to assist a user in interacting with an elevator. For instance, the accessibility system 210 can be configured to obtain sensor data regarding a user 452 and an object 450, such as a button or other control of the elevator (e.g., using sensors 402 that are placed in, on, and/or around the elevator). Further, the location determination circuitry 404 can determine the location of the user relative to the object (e.g., the location of the user's hand relative to a particular button). Further still, the notification circuitry 408 can generate notification to the user to assist the user in locating and interacting with the object 450 (e.g., such that the user can operate the elevator).

As an example, one or more of the systems, methods, and/or computer program products described herein can be configured to assist a user in interacting with a parking meter or vending machine. For instance, the accessibility system 210 can be configured to obtain sensor data regarding a user 452 and an object 450, such as a button or other control of the parking meter or vending machine (e.g., using sensors 402 that are placed in, on, and/or around the parking meter or vending machine). Further, the location determination circuitry 404 can determine the location of the user relative to the object (e.g., the location of the user's hand relative to a particular button). Further still, the notification circuitry 408 can generate notification to the user to assist the user in locating and interacting with the object 450 (e.g., such that the user can operate the parking meter or vending machine).

As an example, one or more of the systems, methods, and/or computer program products described herein can be configured to assist a user in interacting with a computerized kiosk (e.g., using sensors 402 that are placed in, on, and/or around the kiosk). For instance, the accessibility system 210 can be configured to obtain sensor data regarding a user 452 and an object 450, such as a button, mouse, touch screen, or other control of the kiosk. Further, the location determination circuitry 404 can determine the location of the user relative to the object (e.g., the location of the user's hand relative to a particular control). Further still, the notification circuitry 408 can generate notification to the user to assist the user in locating and interacting with the object 450 (e.g., such that the user can operate the kiosk).

The embodiments above are provided as illustrative examples. In practice, the embodiments described herein can be configured to assist a user in interacting with any system or device, either in addition to or instead of those described herein.

In the foregoing description, aspects and embodiments of the present disclosure have been described with reference to numerous specific details that can vary from implementation to implementation. Accordingly, the description and drawings are to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising,” in the foregoing description or following claims, what follows this phrase can be an additional step or entity, or a sub-step/sub-entity of a previously-recited step or entity.

What is claimed is:

1. A method comprising:
obtaining, by an accessibility system of a vehicle, sensor data representing a user;
determining, by the accessibility system, at least one of:
a distance between a body part of the user and an object associated with the vehicle, or
a direction from the body part of the user to the object; and
causing, by the accessibility system, at least one notification to be presented to the user, wherein the least one notification indicates at least one of:
the distance between the body part of the user and the object, or
the direction from the body part of the user to the object.

2. The method of claim 1, wherein the sensor data comprises at least one of:
an image,
a video,
radar data,
LiDAR data, or
ultrasonic sensor data.

3. The method of claim 1, wherein sensor data is obtained from a sensor system of the vehicle, and wherein the sensor system comprises at least one of:
a camera,
a radar sensor,
a LiDAR sensor, or
an ultrasonic sensor.

4. The method of claim 1, wherein the body part of the user comprises at least one of:
a hand of the user,
a finger of the user, or
a foot of the user.

5. The method of claim 1, wherein the object comprises a door handle of the vehicle.

6. The method of claim 1, wherein the object comprises as control mechanism of the vehicle.

7. The method of claim 1, wherein the at least one notification comprises an auditory signal.

8. The method of claim 7, wherein a frequency of the auditory signal varies depending on the distance between the body part of the user and the object.

9. The method of claim 1, further comprising:
continuously determining, until a stop condition is satisfied, at least one of:
the distance between the body part of the user and the object, or
the direction extending from the body part of the user to the object.

10. The method of claim 9, further comprising:
continuously causing, until the stop condition is satisfied, the at least one notification to be presented to the user.

11. The method of claim 9, wherein the stop condition comprises:
a determination that the body part of the user is in contact with the object.

12. The method of claim 9, wherein the stop condition comprises:
a determination that the body part of the user is within a threshold distance from the object.

13. The method of claim 1, wherein causing the at least one notification to be presented to the user comprises:
causing an audio speaker of the vehicle to present the at least one notification.

14. The method of claim 1, wherein causing the at least one notification to be presented to the user comprises:
causing a mobile device associated with the user to present the at least one notification.

15. The method of claim 14, wherein the mobile device comprises at least one of a communications device or a wearable device.

16. A system, comprising:
at least one processor; and
at least one non-transitory storage media storing instructions that, when executed by the at least one processor, cause the at least one processor to:
obtain sensor data representing a user;
determine at least one of:
a distance between a body part of the user and an object associated with a vehicle, or
a direction from the body part of the user to the object; and
cause at least one notification to be presented to the user, wherein the least one notification indicates at least one of:
the distance between the body part of the user and the object, or
the direction from the body part of the user to the object.

17. At least one non-transitory storage media storing instructions that, when executed by at least one processor, cause the at least one processor to:
obtain sensor data representing a user;
determine at least one of:
a distance between a body part of the user and an object associated with a vehicle, or
a direction from the body part of the user to the object; and
cause at least one notification to be presented to the user, wherein the least one notification indicates at least one of:
the distance between the body part of the user and the object, or
the direction from the body part of the user to the object.
